(19) United States
(12) Patent Application Publication
(10) Pub. No.: US 2024/0189238 A1
(43) Pub. Date: Jun. 13, 2024

(54) FRUIT AND/OR VEGETABLE BASED COMPOSITIONS AND METHODS OF PREPARING THE SAME

(71) Applicant: The Well Fruit Company, Inc., Tampa, FL (US)

(72) Inventors: James MOSBAUGH, Tampa, FL (US); John LINDERMAN, Wilton, CT (US); Jeffery ROME, Hidden Hills, CA (US)

(21) Appl. No.: 18/225,052

(22) Filed: Jul. 21, 2023

Related U.S. Application Data
(60) Provisional application No. 63/391,458, filed on Jul. 22, 2022.

(51) Int. Cl. A61K 9/19; A61K 9/00

(52) U.S. Cl. CPC A61K 9/19; A61K 9/0053

(57) ABSTRACT

The present disclosure relates to compositions comprising freeze-dried fruit and/or vegetable component and active ingredient wherein the active ingredient is conserved. The present disclosure further relates to methods for preparing the freeze-dried fruit and/or vegetable based compositions for active ingredient delivery.

### CROSS-REFERENCE TO RELATED APPLICATIONS

This application claims the benefit of U.S. Provisional Application No. 63/391,458, filed Jul. 22, 2022, which is incorporated herein by reference.

### BACKGROUND

Oral delivery of an active ingredient is one of the most common and acceptable delivery routes. Delivery systems such as capsules, tablets, softgels, powders or gummies are well defined but there are drawbacks associated with them. Capsules and tablets generally require binders, excipients and excessive processing steps for manufacture which make the products far from natural. In addition, they are sometimes hard for some people to swallow, especially for children and those with difficulty in swallowing or with dysphagia. Softgels are an ideal delivery form for lipid and fat soluble active ingredients, but they can be sensitive to heat and moisture during shelf-life. The physical migration of components between shell, fill and external environment and the physical or chemical reactions (e.g., crosslinking between gelatin chains) of the shell and fill components can result in instability and shell brittleness and insolubility. Powder formulations allow for the product to be mixed into a liquid and consumed as a drink, but active ingredients that are bitter in taste may be difficult to flavor in a powder and the requirement to mix the powder into a liquid before consumption makes it inconvenient. Gummies are an attractive delivery vehicle but the processing typically includes heating the active ingredients with pectin or gelatin and other additives which may potentially degrade the active ingredients.

### SUMMARY

Recognized herein is a need for natural, shelf stable, ready-to-consume, and easy-to-consume compositions in solid dosage form for active ingredient delivery. Further, recognized herein is new and/or improved methods for manufacturing a natural, shelf stable, ready-to-consume, and easy-to-consume composition in solid dosage form for active ingredient delivery.

Provided in various aspects herein are compositions comprising freeze-dried fruit and/or vegetable component and at least one active ingredient. Also provided in various aspects herein are methods of manufacturing such compositions.

In an aspect, provided herein is a method for producing a fruit and/or vegetable based composition, comprising: (a) providing a fruit and/or vegetable component and at least one active ingredient; (b) processing the fruit and/or vegetable component and the at least one active ingredient to generate a mixture; and (c) freeze-drying at least a portion of the mixture to generate the fruit and/or vegetable based composition; wherein (b) is performed at temperatures less than 0 degree Celsius.

In some embodiments, the processing comprises blending the fruit and/or vegetable component and the at least one active ingredient with mixing rate of about 20 to about 200 rpm and pressure of about 750 to about 5500 psi under temperatures less than 0 degree Celsius. In some embodiments, the blending is performed using a blender. In some embodiments, the processing is performed without an increase of temperature of the fruit and/or vegetable based composition above ambient temperature.

In some embodiments, the method further comprises, prior to (c), directing the mixture to a mold, wherein the mold comprises at least one cavity. In some embodiments, the at least one cavity has a dimension of about 1 cm to about 10 cm and has a shape selected from the group consisting of rounded, square, triangular, elliptical, semicircular, ribboned, rectangular, star, heart, or specialty shape. In some embodiments, the method further comprises freeze-drying the mold to form a solid article of the fruit and/or vegetable based composition. In some embodiments, the method further comprises, prior to (c), extruding the mixture to form a solid article. In some embodiments, the extruding is conducted under temperatures less than 0 degree Celsius. In some embodiments, the solid article has a dimension of about 1 cm to 10 cm and has a shape selected from the group consisting of rounded, square, triangular, elliptical, semicircular, ribboned, rectangular, star, heart, or specialty shape. In some embodiments, the method further comprises, prior to (c), freezing the mixture to a temperature less than −20 degrees Celsius.

In some embodiments, the freeze-drying is performed at temperatures between −80 degrees Celsius and 40 degrees Celsius. In some embodiments, the change in temperature during freeze-drying is between 20 degrees Celsius and 80 degrees Celsius. In some embodiments, the freeze-drying is performed by lowering the temperature to between −20 degrees Celsius and −80 degrees Celsius at cooling rate of between 0.5 and 20 degrees Celsius per minute.

In some embodiments, the fruit and/or vegetable based composition is substantially free of preservatives. In some embodiments, the fruit and/or vegetable based composition is substantially free of additives. In some embodiments, the fruit and/or vegetable based composition is substantially free of a binding agent. In some embodiments, the fruit and/or vegetable based composition is orally disintegrable. In some embodiments, the fruit and/or vegetable based composition comprises a water activity of about 0.15 to about 0.40. In some embodiments, the fruit and/or vegetable based composition comprises a water activity of about 0.2 to about 0.35. In some embodiments, the fruit and/or vegetable based composition provides a crunchy mouth feel.

In some embodiments, the at least one active ingredient is selected from the group consisting of a dietary supplement ingredient, pharmaceutical, a prodrug, a homeopathic drug, traditional Chinese medicine, ayurvedic compound, a flavor or fragrance, a botanical extract or a combination thereof. In some embodiments, the method further comprises, in (a), providing an additional ingredient. In some embodiments, the additional ingredient is a dairy or food product.

In some embodiments, the fruit and/or vegetable based composition comprises at least one type of fruit component. In some embodiments, the fruit and/or vegetable based composition comprises at least one type of vegetable component. In some embodiments, the fruit and/or vegetable based composition comprises at least one type of fruit component and at least one type of vegetable component. In some embodiments, the fruit and/or vegetable component is Individually Quick Frozen and Ready to Eat.

In some embodiments, the method further comprises coating the fruit and/or vegetable based composition. In some embodiments, the coating comprises lipids, sweeteners, flavors, emulsifiers, protein, salt, milk product, soy product, sugar, or edible wax.

In another aspect, disclosed herein is a composition, comprising a carrier matrix and at least one active ingredient, wherein the carrier matrix comprises freeze-dried fruit component in solid dosage form.

In some embodiments, the carrier matrix comprises at least one type of freeze-dried fruit component. In some embodiments, the carrier matrix further comprises at least one type of freeze-dried vegetable component. In some embodiments, the freeze-dried fruit component is selected from grape component, strawberry component, kiwi component, banana component, mango component, blueberry component, or pineapple component. In some embodiments, the freeze-dried fruit component is blended at temperatures less than 0 degree Celsius. In some embodiments, the carrier matrix is molded. In some embodiments, the carrier matrix is molded into a shape that is rounded, square, triangular, elliptical, semicircular, ribboned, rectangular, star, heart, or specialty shape. In some embodiments, the carrier matrix is extruded. In some embodiments, the carrier matrix is extruded to a shape that is rounded, square, triangular, elliptical, semicircular, ribboned, rectangular, star, heart, or specialty shape. In some embodiments, the shape has a dimension of about 1 cm to about 10 cm. In some embodiments, the at least one active ingredient is selected from the group consisting of a dietary supplement ingredient, pharmaceutical, a prodrug, a homeopathic drug, traditional Chinese medicine, ayurvedic compound, a flavor or fragrance, a botanical extract or a combination thereof. In some embodiments, the at least one active ingredient is probiotic. In some embodiments, the carrier matrix is substantially free of preservatives. In some embodiments, the carrier matrix is substantially free of additives.

In yet another aspect, provided herein is a composition consisting essentially of a carrier matrix and at least one active ingredient, wherein the carrier matrix consists essentially of at least one type of fruit component.

In some embodiments, the composition further consists essentially of at least one type of vegetable component.

In some embodiments, the at least one active ingredient is between 1 wt % and 40 wt % of the composition. In some embodiments, the carrier matrix has the at least one active ingredient homogenously distributed throughout the carrier matrix. In some embodiments, the at least one active ingredient is selected from the group consisting of a dietary supplement ingredient, pharmaceutical, a prodrug, a homeopathic drug, traditional Chinese medicine, ayurvedic compound, a flavor or fragrance, a botanical extract or a combination thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein) of which:

FIG. 1 illustrates exemplary water activity of a variety of freeze dried fruits with different active ingredient.

FIG. 2 illustrates exemplary water activity of a variety of freeze dried fruits with different active ingredient and dimensions of the final product.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used in the specification and claims, the singular forms “a,” “an,” and “the” can include plural references unless the context clearly dictates otherwise.

The term “about” or “approximately,” as used interchangeably herein, generally refers to within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value.

The term “water activity,” as used herein, refers to the amount of free or unbound water that is available in food or equivalent product. It is a measurement of the water that is not bound to components in the food or equivalent product such as sugar, salt or protein. It is measured in values from 0.0 to 1.0. The lower the water activity value, the more “dry” a food or equivalent product is considered. Water activity affects the physical properties of food or equivalent product. Food or equivalent product with a high water activity has a texture that is moist, juicy, tender or chewy. Food or equivalent product with low water activity has texture attributes of crispy or crunchy.

The term “Individually Quick Frozen” and “IQF,” as used interchangeably herein, refers to a process that each individual piece of food is frozen separately from all the others and is frozen very quickly. It prevents big ice crystals from forming inside the cells of the fruit or vegetable.

The term “Ready to Eat” and “RTE,” as used interchangeably herein, refers to a type of food that is ready to eat. It does not require re-heating, re-cooking, or re-processing before serving.

The term “colony forming unit” and “CFU,” as used interchangeably herein, refers to a unit commonly used to estimate the concentration of microorganisms in a test sample.

Provided in various aspects herein are compositions comprising freeze-dried fruit and/or vegetable component and at least one active ingredient. Also provided in various aspects herein are methods of manufacturing such compositions.

In an aspect, the present disclosure provides a method for producing a freeze-dried fruit and/or vegetable based composition, comprising: (a) providing a fruit and/or vegetable component and at least one active ingredient; (b) processing the fruit and/or vegetable component and the at least one active ingredient to generate a mixture at temperatures less than 0 degree Celsius; and (c) freeze-drying at least a portion of the mixture to generate a freeze-dried fruit and/or vegetable based composition.

In an aspect, the freeze-dried fruit and/or vegetable based composition comprises freeze-dried fruit and/or vegetable as a carrier matrix and the at least one active ingredient homogenously distributed throughout the carrier matrix.

In some embodiments, the fruit component may comprise fresh, frozen, canned, dried, or pasteurized fruit. In some embodiments, the fruit component may comprise a part, a whole, cut, chopped, pureed, or cooked fruit. In some embodiments, the fruit component may be Individually Quick Frozen and Ready to Eat.

In some embodiments, the fruit component may be any suitable fruit component or combination of fruit components. In some embodiments, the fruit may comprise berries (e.g., strawberries, raspberries, blackberries, blueberries, goji berries, cranberries, boysenberries, elderberries), citrus (e.g., oranges, lemons, grapefruits, limes, pomelos), grapes, apples, bananas, pineapples, cherries, plums, currants, kiwis, mangos, papaya, peaches, nectarines, kumquats, rhubarb, avocado, pears, apricots, plantains, figs, dates, beets, jackfruit, or dragon fruit. In some embodiment, the fruit comprises strawberries, red grades, and bananas. In some embodiment, the fruit comprises strawberries, bananas, and blueberries. In some embodiment, the fruit comprises mangos, red grapes, pineapples, and bananas.

In some embodiments, the vegetable component may comprise fresh, frozen, canned, dried, or pasteurized vegetable. In some embodiments, the vegetable component may comprise a part, a whole, cut, chopped, pureed, or cooked vegetable. In some embodiments, the vegetable component may be Individually Quick Frozen and Ready to Eat.

In some embodiments, the vegetable component may be any suitable vegetable component or combination of vegetable components. In some embodiments, the vegetable may comprise asparagus, leeks, bell peppers, hot peppers, parsnips, broccoli, Brussel sprouts, cabbage, cauliflower, kale, soybeans, peas, swede, chilis, squash, corn, tomatoes, water chestnuts, bamboo shoots, ginger, carrots, sweet potatoes, potatoes, pumpkin, courgette, onions, garlic, shallots, celery, green beans, olives, or spinach.

In some embodiments, the composition may comprise at least one type of fruit component. In some embodiments, the composition may comprise at least one type of vegetable component. In some embodiments, the composition may comprise at least one fruit component and at least one vegetable component.

In some embodiments, the active ingredient may comprise, but is not limited to dietary supplements, over the country counter drugs, pharmaceuticals including drugs and prodrugs, biological therapies, traditional Chinese medicine ingredients, Ayurvedic medicine ingredients, or other active or therapeutic ingredients for human and/or animal health.

Non-limiting examples of dietary supplements are selected from the group consisting of prebiotics (e.g., inulin, fructo-oligosaccharides, galacto-oligosaccharides), probiotics, minerals (e.g., calcium, phosphorus, potassium, sodium, magnesium, iron, zinc, iodine, sulfur, cobalt, copper, fluoride, manganese, selenium, chromium, molybdenum, boron, silica), vitamins (e.g., vitamin A, vitamin B1, vitamin B2, vitamin B3, vitamin B5, vitamin B6, vitamin B12, biotin, folate, folic acid, vitamin C, vitamin D3, vitamin E, vitamin K2), herbs or botanicals or plant extracts (e.g., Ashwagandha, ginseng, maca, tongkat ali, licorice root, psyllium, fiber, adaptogens, thyme, phytonutrients, lutein, phytosterols, quercetin, green tea, green coffee extract, guarana, monolaurin, echinacea, turmeric, baicalin, capsaicin, kelp, oleuropein), amino acids (e.g., L-theanine, L-tryptophan, taurine, alanine, isoleucine, leucine, methionine, valine, tyrosine, glutamine, asparagine, cysteine, serine, threonine, aspartic acid, glutamic acid, arginine, histidine, lysine, glycine, proline, N-acetyl cysteine), oil supplements (e.g., cod liver oil, primrose oil), polyunsaturated fatty acids (e.g., Omega-3, fish oil, krill Oil,), antioxidants (e.g., Coenzyme Q10, alpha lipoic acid, lycopene, zeaxanthin, choline), hormones (e.g., melatonin), enzymes, or proteins (e.g., collagen protein).

Non-limiting examples of over the counter drug ingredients may comprise analgesics, antacids, antithelminic, anticaries, antidiarrheal, antiemetic, antiflatulent, antifungal, antimicrobial, cholecystokinetic, cough/cold, daytime sedative, digestive aid, emetic, gingivitis/plaque, internal analgesic, laxative, menstrual/diuretic, nighttime sleep aid, other oral health care, overindulgence, stimulant, weight control, or other over the counter drug.

Non-limiting examples of pharmaceutical ingredients may comprise antipyretics, analgesics, antimalarial drugs, antibiotics, antiseptics, mood stabilizers, hormone replacements, oral contraceptives, stimulants, tranquilizers, antianxiety, cardiovascular drugs such as beta-receptor blockers, calcium channel blockers, diuretics, cardiac glycosides, antiarrhythmics, nitrates, antianginals, vasoconstrictors, vasodilators, HMG-CoA reductase inhibitors (statins) and other hypolipidaemic agents, coagulation or anticoagulant drugs such as heparin, antiplatelet drugs, fibrinolytics, anti-hemophilic drugs, homeostatic drugs, antihypertensive drugs such as ACE inhibitors, angiotensin, ACE inhibitors, angiotensin receptor blockers, beta-blockers, a blockers, calcium channel blockers, thiazide diuretics, loop diuretics, aldosterone inhibitors, antibiotic ingredients such as penicillins, cephalosporins, macrolides, fluoroquinolones, sulfonamides, tetracyclines, or aminoglycosides.

In some embodiments, the active ingredient may comprise probiotics.

In some embodiments, the at least one active ingredient may be between 1 wt % and 40 wt % of the freeze-dried fruit and/or vegetable based composition.

In some embodiments, the freeze-dried fruit and/or vegetable based composition may comprise essentially fruit and/or vegetable component, and active ingredient. In some embodiments, the freeze-dried fruit and/or vegetable based composition may be substantially free of additives, binding agents or preservatives.

In some embodiments, the freeze-dried fruit and/or vegetable based composition may comprise an additional ingredient. Non-limiting examples of the additional ingredient may comprise, but are not limited to, a dairy or food product.

The blending of fruit and/or vegetable component with the active ingredient(s) may be done in any suitable manner. In some embodiments, the blending may be performed with a (e.g. commercial) blender, juicer, planetary mixer, pulper, food processor, extruder, certain types of milling tools, static inline mixers, dynamic inline mixer, simple propeller mixers with rotor stator blades, or dough mixer.

The blending of fruit and/or vegetable component with the active ingredient(s) may be performed at a temperature less than 0° C. In some embodiments, the blending may be performed at a temperature less than −1° C., less than −2° C., less than −3° C., less than −4° C., less than −5° C., less than −6° C., less than −7° C., less than −8° C., less than −9° C., less than −10° C., less than −11° C., less than −12° C., less than −13° C., less than −14° C., less than −15° C., less than −16° C., less than −17° C., less than −18° C., less than −19° C., less than −20° C. In some embodiments, during the blending, the temperature may not increase above ambient temperature. In general, cold blending can provide the advantage of preserving the fruit component, vegetable component, and/or the active ingredient.

In some embodiments, the blending may be done with mixing rate of about 20 to about 200 rpm. In some embodiments, the blending may be done under pressure of about 750 to about 5500 psi.

In some embodiments, the blending may be performed with the mixer in a freezer. In some embodiments, the freezer may be a walk-in freezer.

In some embodiments, after the blending, the mixture may be frozen to a temperature less than −20° C. before transferring to a freeze-dryer.

In some embodiments, after the blending, the mixture may be molded, such as into a desired form. In some embodiments, the mixture may be directed to a mold, such as a mold with cavities. The cavities may have a shape or a plurality of shapes. Non-limiting shape of the cavities may comprise, but is not limited to, rounded, square, triangular, elliptical, semicircular, ribboned, rectangular, star, heart, or specialty shape. The cavities may have a dimension of about 1 cm to about 10 cm. Molds with cavities may be made from any appropriate food grade material including, but not limited to, stainless steel, aluminum, silicone, and food grade plastics such as HPDE, LDPE, PE, PP, ABS, PEEK. Molds with cavities may also be equipped with a pneumatic release system that employs a manifold leading to a plurality of small holes at the base of the cavities wherein once the articles are ready to be removed from the mold, they can be gently blown from the mold using compressed air or other gas. The gas may be a reducing gas that has a direct impact on the surface chemistry, appearance, flavor and other organoleptic properties of the article. The mixture may be directed to the mold by scooping, pumping, extrusion, or pouring.

Alternatively, the mixture may be extruded to a tray or any collector through an extruder. The extruder outlet may be configured to any shape. Non-limiting shape of extruder outlet may comprise, but is not limited to rounded, square, triangular, elliptical, semicircular, ribboned, rectangular, star, heart, or specialty shape. The extruder outlet may have a dimension of about 1 cm to about 10 cm.

In some embodiments, the extrusion may be conducted under a temperature less than 0° C., less than −1° C., less than −2° C., less than −3° C., less than −4° C., less than −5° C., less than −6° C., less than −7° C., less than −8° C., less than −9° C., less than −10° C., less than −11° C., less than −12° C., less than −13° C., less than −14° C., less than −15° C., less than −16° C., less than −17° C., less than −18° C., less than −19° C., or less than −20° C.

In some embodiments, the mold, the tray or the collector may be placed in a freeze dryer. The freeze dryer may be set to cool down at a cooling rate of about 0.5 to about 20° C. per minute. When the mixture reaches a temperature less than −20° C., less than −25° C., less than −30° C., less than −35° C., less than −40° C., less than −45° C., less than −50° C., less than −55° C., less than −60° C., less than −65° C., less than −70° C., less than −75° C., or less than −80° C., a vacuum may be pulled to the freeze dryer. Alternatively, the vacuum can be pulled before or during the cooling process. After the vacuum reaches about 10⁻³ to about 10⁻⁴ Torr, the freeze dryer may be allowed to heat and increase the temperature slowly at a heating rate of about 0.01 to about 5° C. per minute to a temperature less than 40° C., less than 35° C., less than 30° C., less than 25° C., less than 20° C., less than 15° C., less than 10° C., less than 5° C., or less than 0° C. The mixture may be kept at the final temperature for about 0.5 h to about 72 h. Alternatively, the mixture may be heated rapidly to a temperature less than 20° C., less than 15° C., less than 10° C., less than 5° C., or less than 0° C., and maintained at the temperature for about 0.5 h to 72 h.

In some embodiments, the temperature change during the drying process may be from about 20° C. to about 80° C.

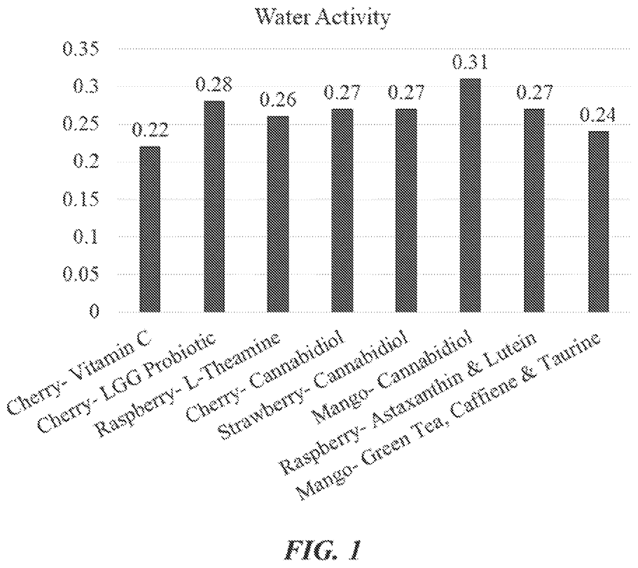

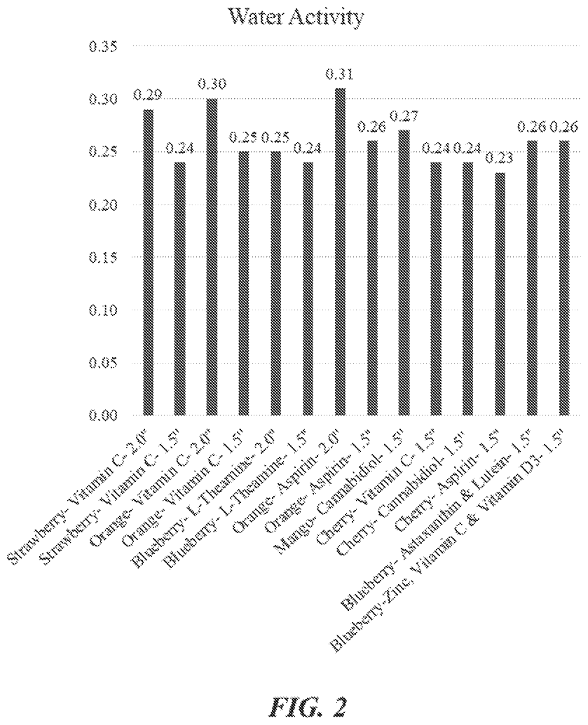

Through the freeze-drying process, solid water molecules in the mixture sublime to gaseous water and are subsequently removed from the freeze dryer by vacuum. Referring to FIGS. 1 and 2, the water activity of exemplary freeze-dried fruit and/or vegetable based compositions may be from about 0.2 to about 0.35. In some embodiments, the water activity may be from about 0.15 to about 0.40.

In some embodiments, the freeze-dried fruit and/or vegetable based composition may have a hardness of about 4.0 newtons (N) to about 15 N. In some embodiments, the freeze-dried fruit and/or vegetable based composition may have a hardness of about 4.0 N to about 9.5 N.

In some embodiments, the freeze-dried fruit and/or vegetable based composition may have a crunchy mouth feel.

In some embodiments, the freeze-dried fruit and/or vegetable based composition may be orally disintegrable.

In some embodiments, the freeze-dried fruit and/or vegetable based composition may comprise a coating. Non-limiting examples of the coating may comprise, but are not limited to, lipids, sweeteners, flavors, emulsifiers, protein, salt, milk product, soy product, sugar, or edible wax. In some embodiments, the coating may also be fortified with other active ingredients including supplement ingredients. In some embodiments, the coating may be applied to protect the freeze-dried fruit and/or vegetable based composition from ambient moisture. In some embodiments, the coating may be used as an inclusion composition in a food that is meant to be hydrated or mixed with a liquid prior to consuming, such as cereal and milk. The coating may be applied by enrobing, panning, spraying, dipping or other methods.

In some embodiments, the at least one active ingredient in the fruit and/or vegetable based composition may be conserved to a high degree due to the cold process and gentle sublimation, ensuring that the at least one active ingredient does not need over-dosing which is often needed in the production of dietary supplements that operates at room temperature or employs heat.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Freeze-dried Fruit Based Composition Comprising Probiotics

Previously washed and frozen blueberries (35-45 wt %), pineapple (12-22 wt %), grapes (15-30 wt %), and bananas (10-20 wt %) were weighed, bulk mixed, and fed into a commercial juicer with the pulp separator and any screen removed. The juicer consists of a tube containing a cylinder with variable speed. The distance from the outer circumference of the cylinder and interior circumference of the tube controls the effective barrel pressure of the extruder. The fruits were blended at a speed of approximately 200 rpm and a pressure of 4000 psi under a temperature of about −5 to 0° C. The fruit mixture was then slowly added through a hopper with a plunger to an extrusion barrel. The resultant slurry of fruits was collected in another mixing vessel wherein the active probiotic powder (LGG Lactobacillus rhamnosus probiotic, about 2 wt %) was added. The mixing vessels was placed under a commercial dough mixer and mixed for time sufficient to adequately disperse the active probiotic through the fruit mixture as homogeneously as possible to generate a fruit-probiotic mixture.

The fruit-probiotic mixture was transferred to trays with cavities to create a plurality of articles with a weight of approximately 2.1 grams per article and an initial load of approximately 2.77 billion CFU probiotic per article (assayed via flow cytometry and direct culture to enumerate the CFUs). The trays were transferred directly to the freeze dryer or held in a −60° C. freezer until ready for processing.

Once the trays were transferred to the freeze dryer, the freeze dryer was set to run a 48 hour cycle. The chamber containing the trays with the fruit-probiotic mixture was cooled to −60° C. at a rate of about 1-3° C. per minute. Once the temperature of the fruit-probiotic mixture, not the chamber, reached −60° C., a vacuum pump was turned on and the pressure in the chamber was quickly dropped to between 10⁻³ to 10⁻⁴ Torr to facilitate the sublimation of the frozen water from the fruit-probiotic mixture to the collection condenser. Minimum pressures were achieved within 20-60 minutes.

As soon as an appropriate vacuum environment in the chamber was created, the temperature was set to increase from −60° C. to −10° C. over 24 hours. The slow increase in temperature ensured that nearly 100% of the loaded probiotic was conserved in the product, and not lost to heat, excessive contact with ambient oxygen, or high shear processes that could have degraded it. The product was collected and tested for water activity, moisture, brix, organoleptics, density and dissolution to the specification.

The resultant product was a rounded probiotic dietary supplement having a diameter of about 1.27 cm (½ inch) and a weight of about 0.444 grams. The product had a brix of about 0.26 to 0.29. Upon assay of the article via flow cytometry, the CFU value was shown to be 2.3 billion. The probiotic was well conserved during the process.

### Example 2: Freeze-Dried Fruit Based Composition Comprising Probiotics

A 2000 g fruit mixture of about 50 wt % strawberries, about 30 wt % bananas, and about 20 wt % grapes was macerated via waring blender. About 50 grams of probiotic powder was added to 500 grams of the fruit mixture and blended using commercial dough mixer for 10 minutes. Next, the remaining 1500 grams of the fruit mixture was added to the dough mixer and mixed for another 10 minutes to generate a fruit-probiotic mixture. This fruit-probiotic mixture was then transferred to small trays with cavities to form approximately 1.5 cm×1.5 cm×1.5 cm cubes. The trays were sent through a liquid nitrogen freeze tunnel and frozen to −60° C. The cubes were removed and placed into a freeze dryer for freeze drying. In the freeze dryer, the temperature of the product was held at −60° C. and then raised in a controlled manner to 20° C. with vacuum down to 10⁻³ Torr and held for 9 hours.

The resultant product was a probiotic dietary supplement containing 5 billion CFU of probiotic delivered in a 100% fruit matrix.

### Example 3: Freeze-Dried Fruit Based Composition Comprising Cannabidiol

A frozen fruit blend comprised of about 47 wt % mangos, about 34 wt % red grapes, about 15 wt % pineapples, and about 4 wt % bananas was made into a slurry by processing with an Omega Juicer at a temperature about −3 to −2° C. To that slurry, a cannabidiol isolate (about 1.2 wt % of the total mixture) was added. This slurry was kept at between −7 and −1° C. while being transferred into molds with 40 cavities that hold about 2.1 grams of the fruit-cannabidiol slurry in each cavity. The loaded trays were packed in dry ice to instantly freeze for storage. After about 20 minutes to 1 hour, the pieces were removed from the molds and transferred to Labconco® Bench Sample Prep Lyophilizer. The pieces were freeze dried by dropping the temperature to −60° C., pulling a 10⁻³ Torr vacuum, and slowly allowing the drier to reach room temperature (approximately 3 days).

The resultant pieces were small marble shaped fruit pieces weighing an average of 500 mg. The water activity was less than 0.2. Each piece contained about 25 mg of cannabidiol.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

1-50. (canceled)

51. A method for producing a fruit and/or vegetable based composition, comprising:
(a) providing a fruit and/or vegetable component and at least one active ingredient;
(b) processing said fruit and/or vegetable component and said at least one active ingredient to generate a mixture; and
(c) freeze-drying at least a portion of said mixture to generate said fruit and/or vegetable based composition, wherein (b) is performed at temperatures less than 0 degree Celsius.

52. The method of claim 51, further comprising, prior to (c), directing said mixture to a mold, wherein said mold comprises at least one cavity comprising a dimension of about 1 cm to about 10 cm and a shape selected from the group consisting of rounded, square, triangular, elliptical, semicircular, ribboned, rectangular, star, heart, or specialty shape.

53. The method of claim 52, further comprising freeze-drying said mold to form a solid article of said fruit and/or vegetable based composition.

54. The method of claim 51, further comprising, prior to (c), extruding said mixture to form a solid article, wherein said extruding is conducted under temperatures less than 0 degree Celsius.

55. The method of claim 54, wherein said solid article has a dimension of about 1 cm to 10 cm and has a shape selected from the group consisting of rounded, square, triangular, elliptical, semicircular, ribboned, rectangular, star, heart, or specialty shape.

56. The method of claim 51, further comprising, prior to (c), freezing said mixture to a temperature less than −20 degrees Celsius.

57. The method of claim 51, wherein said freeze-drying is performed at temperatures between −80 degrees Celsius and 40 degrees Celsius.

58. The method of claim 57, wherein a change in temperature during freeze-drying is between 20 degrees Celsius and 80 degrees Celsius.

59. The method of claim 51, wherein said fruit and/or vegetable based composition is substantially free of preservatives, additives, and/or a binding agent.

60. The method of claim 51, wherein said fruit and/or vegetable based composition is orally disintegrable and provides a crunchy mouth feel.

61. The method of claim 51, wherein said fruit and/or vegetable based composition comprises a water activity of about 0.15 to about 0.40.

62. The method of claim 51, wherein said processing comprises blending said fruit and/or vegetable component and said at least one active ingredient with mixing rate of about 20 to about 200 rpm and pressure of about 750 to about 5500 psi under temperatures less than 0 degree Celsius.

63. The method of claim 62, wherein said blending is performed using a blender.

64. The method of claim 51, wherein (c) is performed by lowering the temperature to between −20 degrees Celsius and −80 degrees Celsius at cooling rate of between 0.5 and 20 degrees Celsius per minute.

65. The method of claim 51, further comprising, in (a), providing an additional ingredient, wherein said additional ingredient is a dairy or food product.

66. The method of claim 51, wherein said at least one active ingredient is selected from the group consisting of a dietary supplement ingredient, pharmaceutical, a prodrug, a homeopathic drug, traditional Chinese medicine, ayurvedic compound, a flavor or fragrance, a botanical extract or a combination thereof.

67. The method of claim 51, wherein (b) is performed without an increase of temperature of said fruit and/or vegetable based composition above ambient temperature.

68. The method of claim 51, wherein said fruit and/or vegetable based composition comprises at least one type of fruit component and/or at least one type of vegetable component.

69. The method of claim 51, wherein said fruit and/or vegetable component is Individually Quick Frozen and Ready to Eat.

70. The method of claim 51, further comprising coating said fruit and/or vegetable based composition, wherein said coating comprises lipids, sweeteners, flavors, emulsifiers, protein, salt, milk product, soy product, sugar, or edible wax.
